(19) United States
(12) Patent Application Publication
(10) Pub. No.: US 2026/0020865 A1
(43) Pub. Date: Jan. 22, 2026

(54) CUTTING BLOCK

(71) Applicants: Smith & Nephew, Inc., Memphis, TN (US); Smith & Nephew Orthopaedics AG, Zug (CH)

(72) Inventors: Thomas Joseph Francis, Cordova, TN (US); Ruxandra Cristiana Marinescu Tanasoca, Memphis, TN (US)

(21) Appl. No.: 19/272,670

(22) Filed: Jul. 17, 2025

Related U.S. Application Data
(60) Provisional application No. 63/672,999, filed on Jul. 18, 2024.
(60) Provisional application No. 63/703,375, filed on Oct. 4, 2024.

(51) Int. Cl. A61B 17/15; A61F 2/38; A61F 2/46; B33Y 80/00

(52) U.S. Cl. CPC A61B 17/155; A61F 2/4684; B33Y 80/00; A61F 2/3859

(57) ABSTRACT

A low-cost disposable. 3-D printed cutting guide. The cutting guide may be made with modular attachment spikes. The cutting guide may be provided in connection with a femoral trial for use in preparing a patient's bone to receive an implant.

This application claims priority to U.S. Provisional Application Ser. No. 63/672,999, filed Jul. 18, 2024, and titled “Cutting Block,” as well as priority to U.S. Provisional Application Ser. No. 63/703,375, filed Oct. 4, 2024, and titled “3D Printed Disposable Femoral Box Cutting Guide,” the entire contents of each of which are incorporated herein by reference.

### FIELD OF THE DISCLOSURE

The present disclosure relates generally to a cutting block for total knee arthroplasty (TKA). In specific embodiments, the cutting block is a 3D-printed, disposable cutting block. The cutting block can also be provided with modular attachment spikes that can be installed and removed from the cutting block as dictated by the surgical method to be used.

### BACKGROUND OF THE DISCLOSURE

Many surgical procedures require preparation of a bone surface to receive an implant. For total knee replacement (TKR) surgery, multiple cuts may need to be made at the end of the distal femur, proximal tibia, and patella to prepare the bone to receive an implant. These cuts are typically made through cutting slots or channels in a cutting block that is fixed to the bone prior to resection.

The industry has tried different designs of total knee arthroplasty cutting blocks. Specifically, it has tried various femoral cruciate retaining (CR), posterior stabilized (PS) and bi-cruciate stabilized (BCS) cutting guides, with one or multiple cutting slots, and different materials, metal, plastic or a combination of materials. Reusable cutting blocks made of metal wear over time and create unwanted metal debris left in the joint. Plastic injection moulded cutting blocks have also left unwanted debris. 3D printed nylon (PA-12) cutting blocks have been used for proximal and distal bone cuts. Surgeons are generally comfortable with the removal of any debris that these may create, because nylon is biocompatible and floats. However, using nylon material for AP cutting blocks introduces other challenges related to stability of the blocks, due to the material being lighter than cutting blocks made of metal. Fixation of the cutting block, particularly the lighter cutting blocks, with respect to the bone is important in order to prevent the block from moving during the resection of bony tissue and introducing cutting errors. At the same time, it is important to have the rigidity of the inner femoral AP box to determine whether the cuts made are correct.

One aim of this disclosure is to create a disposable, low cost, cutting guide that will not generate metal debris and that can help maintain stability and rigidity for accurate cutting. Another aim of this disclosure is the provide a modular attachment system for the cutting block. A further aim is to provide a 3D-printed disposable, low cost, CR (cruciate-retaining) or PS (posterior stabilized) or BCS (bi-cruciate stabilized) cutting guide/femoral trial.

### BRIEF DESCRIPTION OF THE DRAWINGS

By way of example, specific examples of the disclosed device will now be described, with reference to the accompanying drawings, in which:

FIG. 1 shows a side perspective view of a cutting guide system described herein, with a cutting block and a modular attachment spike.

FIG. 2 shows a top plan view of the cutting guide of FIG. 1.

FIG. 3 shows a bottom perspective view of the cutting guide of FIG. 1.

FIG. 4 shows a bottom perspective view of a cutting block having modular finned spikes positioned therein.

FIG. 5 shows a side cross sectional view of an alternate cutting block.

FIG. 6 shows a side perspective view of an alternate spike.

FIG. 7 shows a top perspective view of a cutting block with varied pin openings.

FIG. 8 shows a top perspective view of an alternate cutting block with varied pin openings.

FIGS. 9-11 show alternate external shapes of cutting guide options. For example, it is possible to provide a larger head, alternate fixation holes (positioned more anterior), and varied cutting slot options.

FIG. 12 shows a cutting block with extra material added (where circled) to provide additional stability to the anterior chamfer cut. The slot is partially captured.

FIG. 13 shows a side perspective view of a femoral trial body and box guide that are united together as one unitary instrument.

FIG. 14 shows an anterior view of FIG. 13.

FIG. 15 shows an alternate side view of FIG. 13.

FIG. 16 shows a side perspective view of a trial body and box guide with a slotted section.

FIG. 17 shows an anterior view of FIG. 16.

FIG. 18 shows a side plan view of FIG. 16

FIG. 19 shows a side perspective view of a 3-D printed femoral trials without the box guide.

FIG. 20 shows an anterior view of FIG. 19.

FIG. 21 shows a side plan view of FIG. 19.

FIG. 22 shows an alternate cutting block with openings.

FIG. 23 shows an undersurface (bone-facing surface) of the cutting guide of FIG. 22 showing gullies associated with the openings to assist with placement.

The drawings are not necessarily to scale. The drawings are merely representations, not intended to portray specific parameters of the disclosure. The drawings are intended to depict various examples of the disclosure, and therefore are not considered as limiting in scope. In the drawings, like numbering represents like elements.

### DETAILED DESCRIPTION

Various features or the like of a cutting guide system will now be described more fully herein with reference to the accompanying drawings, in which one or more features of the instrumentation set will be shown and described. It should be appreciated that the various features may be used independently of, or in combination, with each other.

The present disclosure is generally directed to cutting guides and surgical methods for use in knee arthroplasty. Particular embodiments are designed for use in surgically preparing the distal femur or proximal tibia for surgical implantation of a prosthetic knee component. The disclosed cutting block provides stability with the goal of minimizing cutting errors and errors in alignment and preventing re-orientation of the cutting block for successive surgical cuts.

Conventional cutting guides are reusable instruments, usually made of metal. The cutting guide is typically secured to the bone with bone pins, and a sawblade is oscillated in a planar direction and through cutting slots in the guide. A plurality of bone resections are typically made: anterior and posterior cuts, and anterior and posterior chamfer cuts. In some instances, the sawblade can deviate from its intended direction because of saw blade vibration and because its movement is not constrained to one direction. The blade can toggle or flex during normal operation due to the flexibility of the sawblade, cutting technique or bone quality. Because of this, metallic debris generation during bone resection in total knee arthroplasty can create issues. For example, metal micro-particles generated from saw blades, metallic cutting guides, drilling, etc. during total knee arthroplasty can induce a pathogenic tissue response, leading to osteolysis and implant loosening. Cutting errors can impact the fixation and positioning of implants, which can also lead to implant loosening and failure.

Additionally, reusable metallic cutting blocks can become less accurate and precise over time, due to wear of the cutting surfaces. Slots manufactured with a high tolerance are expected to wear over time and can lead to imprecise cuts. Metallic cutting guides can also be costly due to material, tooling, and machining the high precision cutting guide. Cleaning and sterilization time further add to the costs of reusable instruments.

Further, high costs for metallic instruments is another challenge. The material, the tooling, and the need to machine cutting features with high precision can raise the cost of a metallic part no matter what manufacturing process is used. Shipping, cleaning, and sterilization required for reusable metal instruments can also add to the high cost.

Various non-metallic materials have been used to develop cutting guides with various cutting slots. For example, polymeric materials and other low cost and light-weight materials, such as ceramics, ceramic inserts, metallic inserts with plastic bodies, have been explored in an attempt to reduce or eliminate generation of metallic particles during surgery. Plastic injection molding cutting blocks are usually low cost, single use, have a simplified design, and can have one to five cutting slots. Injection molding or insert molding such blocks can be challenging (holding the blades, shrinking, delamination, etc.), as well as expensive if second operations are required. Monolithic ceramic cutting guides can be expensive and prone to brittle failure.

The present disclosure thus relates to a cutting block that is 3D-printed. The cutting block can be 3D printed from various types of biocompatible materials. In some examples, a single material is used. In other examples, a mixture of materials may be used. For example, the main cutting block body may be of nylon, polyethylene, BioMed clear, PPSU (Radel), PEI (Ultem), PEEK, PAEK, any other appropriate materials, or any combinations thereof. In one example, the cutting block can be 3D printed entirely of one material in one build. In another example, the cutting block can be 3D printed using multiple powders/materials in one build to form a single layer (multi-material 3D printing).

Providing the cutting block as a 3D-printed cutting block allows it to be a disposable, single-use cutting block. Once the cutting block is used, it can be disposed of. This prevents the need for any type of sterilization of the block for its re-use, to clean, sterilize, wrap, store, and track hundreds of instruments, and eliminates the Central Service/Sterile Processing and Distribution (CS/SPD) bottleneck, which is currently the biggest problem that surgical operating rooms, ambulatory surgery centers, and other operating arenas face. This can reduce time, eliminate sterilization costs, and overall, can save hospital and manpower resources. 3D printing also allows varying shapes and features to be formed in the cutting block that may otherwise be expensive and time consuming or not available with traditional manufacturing methods. 3D printing enables design freedom, creation of complex geometries, from light, porous structures to highly complex and reinforced sections. Additionally, the designs can be specific to the implant inside box geometry, the implant size, and hand (left or right).

The present disclosure also relates to a cutting block that is 3-D printed. In certain embodiments, the cutting block has modular spikes that can be used to secure the block to resected bone. Providing this modularity allows use of the block with or without the spikes. For example, in some surgeries, the spikes may be used. This is often the case with conventional surgeries. In other types of surgeries, the surgeon may prefer to use the cutting block without spikes (and/or use other openings in the block for fixation). This may be more often the case with robotic-assisted surgeries. The spikes may be made of the same material as the body of the cutting block or of a different material, as described in more detail below. Because the cutting block can be 3-D printed from a lighter material than the typical metal blocks, they are lighter and can bend or move around the resected bone more easily. Accordingly, providing removable spikes can help fixation of the block in place, when and if needed. One overall design goal with this disclosure is to provide a cutting block that can be used for conventional surgeries, as well as robotic surgeries.

A further embodiment of this disclosure provides a femoral trial and box guide that are united together as one instrument. Another embodiment contains just the design of the femoral trial (CR, PS, BCS), but without the box guide. A sawblade is placed in the anterior box guide or cutting slot to perform the first resection. Then the saw blade is moved to the posterior cutting slot or box guide to perform the second resection. This will be followed by a chisel passed through the same anterior and posterior box guides. These options are described in more detail below.

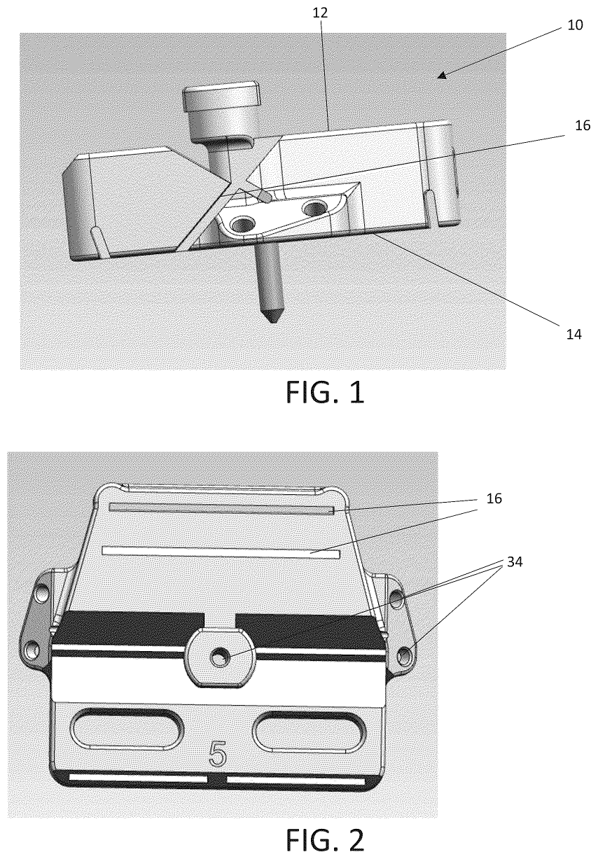

FIGS. 1-8 illustrate exemplary views of a cutting block 10. The cutting block 10 has an upper or top surface 12 and a bone-facing or bottom surface 14. Extending between surfaces 12, 14 are one or more cutting slots 16. Cutting slots 16 are sized and dimensioned to receive a bone saw blade in order to resect the underlying bone to which the cutting block 10 is attached, along desired angles. In the embodiment shown, five cutting 16 slots are present. Such a cutting block could be used to prepare a bone for an implant that has five bone-facing surfaces, one non-limiting example of which is Smith & Nephew's Journey® knee implants. It should be understood that any other number of cutting slots 16 may be provided. In another specific example, one or four cutting slots may be provided. Such a cutting block could be used to prepare a bone for an implant that has four bone-facing surfaces, one non-limiting example of which is Smith & Nephew's Legion® knee implants. It should be understood, however, that any other number of cutting slots 16 may be provided.

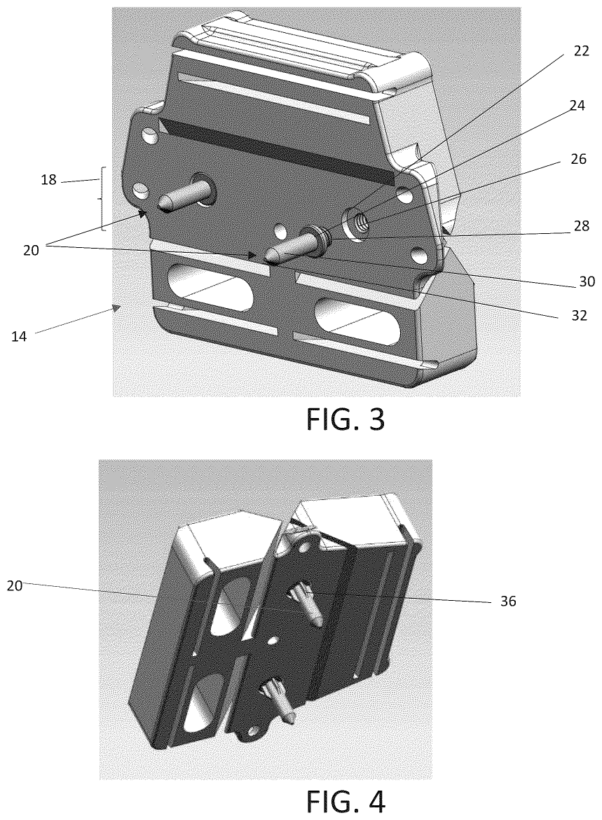

As shown by FIG. 3, the bone-facing surface 14 of the cutting block 10 may have one or more mating features 18. Mating features 18 are sized and configured to receive attachment spikes 20 for enhanced stability. In some examples, the mating features 18 may be formed as indentations 22 in surface 14. The indentations 22 can receive and support a subplate formed as an upper shoulder 24 of a spike 20, described in more detail below. Indentations 22 may be shaped as circular areas that are recessed from the surface 14. The indentations 22 may also have internal threads 26. In this embodiment, each attachment spike 20 may have a corresponding threaded protrusion 28 that can be received by the internal threads 26 for securement of the spike 20 with respect to the block 10. In use, the spike 20 can be screwed into the cutting block 10.

The spike 20 shown by FIG. 3 has a shaft 30, an upper subplate/shoulder 24, and a threaded protrusion 28. The threaded protrusion 28 can extend up from the shoulder 24. This threaded portion 28 can be screwed into the block and cooperate with the internal threads 26. Insertion can be such that the upper shoulder 24 rests within the indentation 22. The subplate/upper shoulder 24 can help distribution of loading during impaction and help avoid point loading which could result in fracture of the cutting block.

At the end of shaft 30 is a pointed end 32, which can be inserted into an opening prepared or made in a patient's bone for securement. It is also possible for the end of the shaft to be blunt. If the surgery to be conducted does not require attachment of the block to the patient's bone via the bone-facing surface 14, then the spikes can be removed from the block (or not inserted in the first place). Use without pins can allow the surgeon to move the block around on the resected femur. For example, the use of certain robotics technologies with sensors may not need spikes. Conventional cases (done without robotics technology) may lend themselves to the use of spikes. Having modular spikes 20 provides a good amount of versatility.

Spikes 20 may have optional fins 36, as shown by FIG. 4. Fins 36 can be shaped as tapered extensions that extend down from the subplate shoulder 24. This can help achieve better fixation and purchase of the block with respect to patient bone.

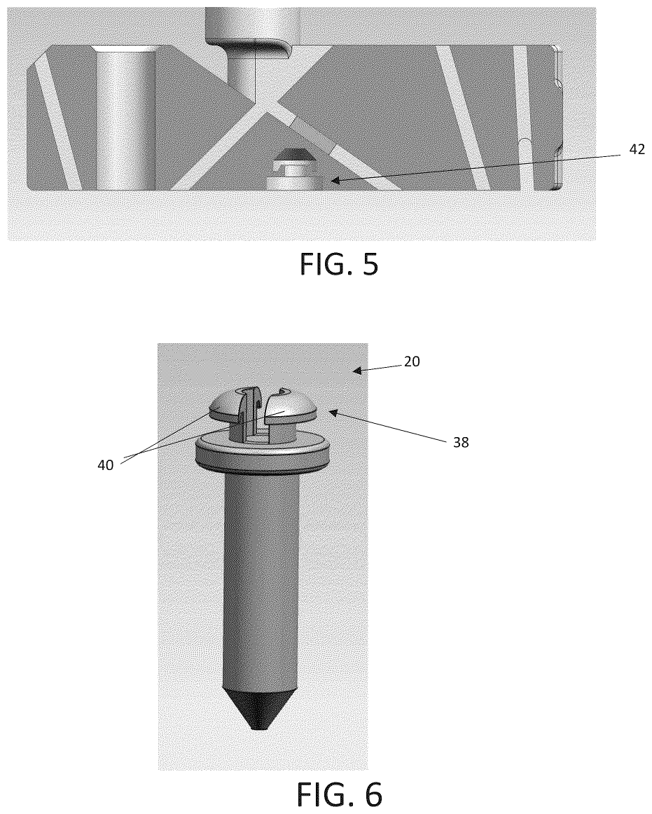

An alternate embodiment mating feature option is shown by FIGS. 5 and 6. In these examples, the spike 20 has a head 38 that is shaped with two arms 40 that can be squeezed together and expand back to their original position. Arms 40 can function similar to a spring-loaded system. The cutting block can have a correspondingly shaped female receiver 42. In use, the arms 40 can be pushed into the receiver 42 and lock the spike 20 into place.

Other connection options are possible. For example, non-limiting example may be a J-hook or dove tail slot, a magnetic securement, twisting, snapping, or any other appropriate connection that can secure two components together in a surgical environment.

Although circular indentations 22 are shown as the receiving area, it is possible for the indentations to take other shapes (e.g., oblong, triangular, square), which can help prevent spikes 20 from rotating with respect to the cutting block. This may be more useful for the mating embodiments that do not use threads that need to turn.

Similar to the cutting block, the modular attachment spikes 20 can also be 3D-printed. For example, they may be manufactured from high impact strength polymers such as PPSU (Radel), PEI (Ultem), PEEK, PAEK, combinations thereof, or other similar materials and or blends with reinforcement fibers such as glass fibers or carbon fibers. The shaft of the spike could have a full or partially printed roughened surface to aid in fixation. Being able to add such a surface coating is one benefit of 3-D printing the spikes. In other examples, the spikes 20 may be manufactured (3D-printed or machined) from metal (for example, if more strength is needed). If desired, a roughened coating may also be applied to machined/metallic spikes.

In a specific example, the block is 3-D printed from a polymer material and the spikes are also 3-D printed from a polymer material. In an alternate example, the block is 3-D printed from a polymer and the spikes are manufactured from metal.

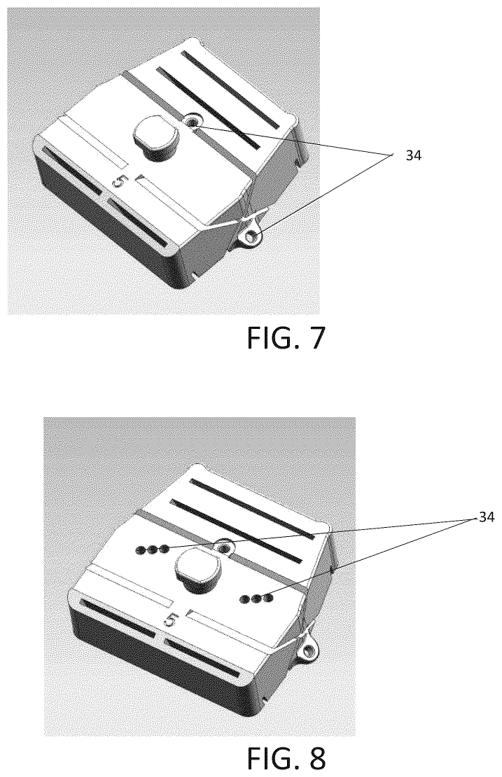

Additional pin holes or openings 34 may be positioned through the cutting block 10 for additional fixation to the bone. For example, the block may be provided with a series of openings 34 (which may also be referred to as pin holes) along the block, which can receive other types of securement features, such as pins. Openings 34 extend from the upper surface 12 to the bone-facing surface 14 of the block 10. Securement features will typically be inserted from the upper surface 12 of the block, extend through the block, and engage underlying bone. FIGS. 1-3 show a central opening 34 and side openings 34. FIGS. 7-8 show alternate positions of openings 34. It should be understood that any combination or any number of openings 34 may be provided. Non-limiting examples of opening placements may be centrally, along block sides, diagonally or otherwise aligned along the upper surface as shown by FIG. 8, sporadically spaced, or elsewhere. Any number of openings may also be provided.

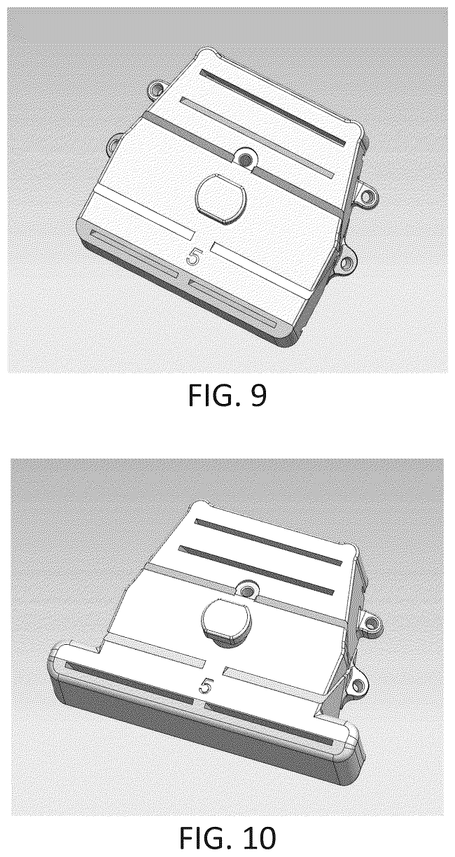

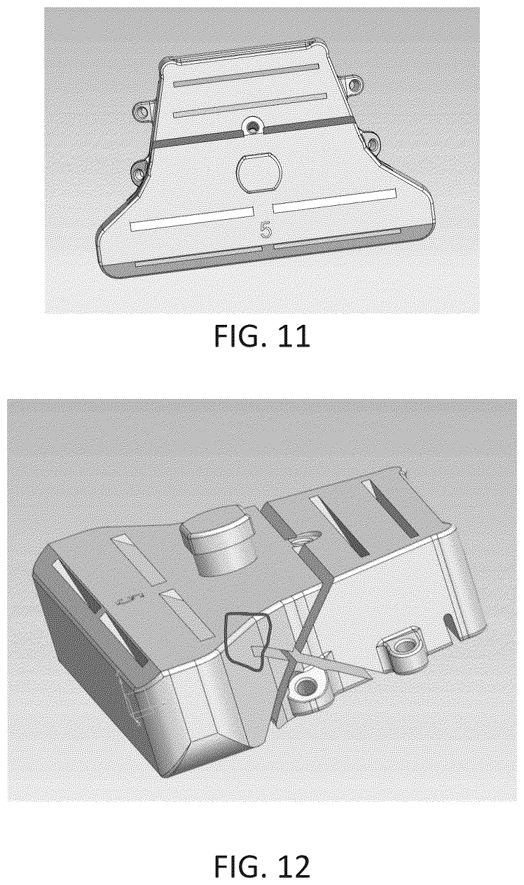

FIGS. 9-11 show alternate external shapes of cutting guide options. For example, it is possible to provide a larger head, alternate fixation holes (positioned more anterior), and varied cutting slot options.

FIG. 12 shows a cutting block with extra material added (where circled) to provide additional stability to the anterior chamfer cut. The slot is partially captured.

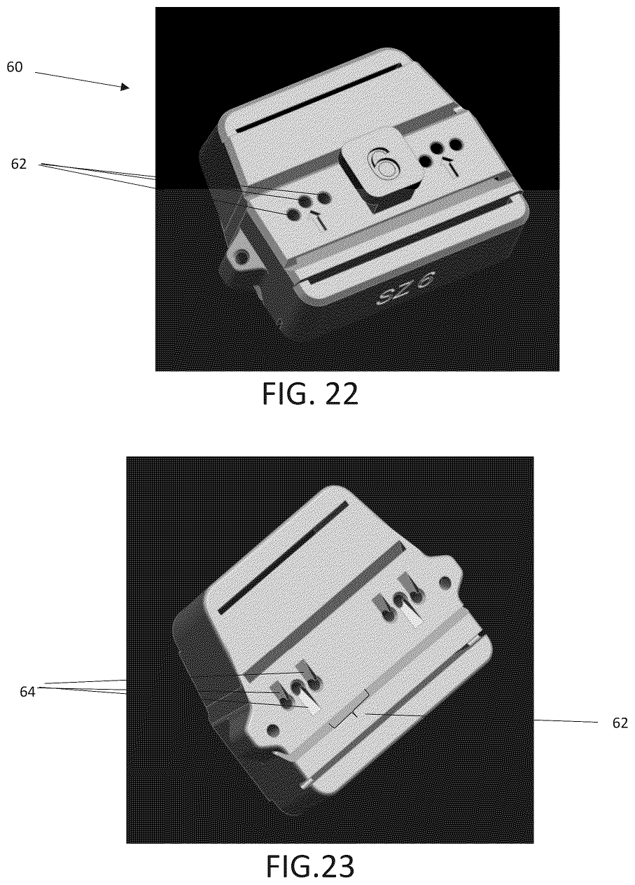

FIGS. 22 and 23 illustrate a spikeless cutting block 60. In this example, the cutting block 60 has a configuration with positional holes 62 for location of the block. Once pins have been placed on the resected distal surface of the femur (which can be done via a conventional femoral sizer or robotic assistance), then the block can be placed. This block embodiment contains gullies 64 on the underside of the block (the bone-facing surface). Gullies 64 can help enable the surgeon to tactically find the holes 62, making it much easier to place the block 60. Gullies can extend just a slight distance away from each opening and form somewhat of an angled groove. This allows the pin that has been placed to gently slide into the opening during placement of the block.

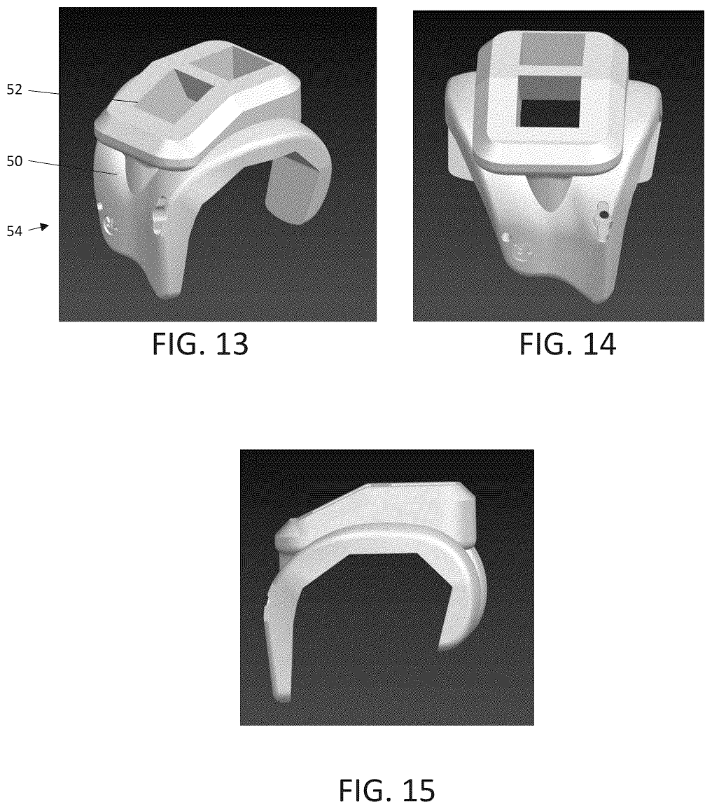

FIGS. 13-21 provide alternate embodiments of cutting blocks that re designed for CR, PS, or BCS surgeries. In FIGS. 13-15, a femoral trial body 50 and box guide 52 are united together as one unitary instrument 54. The trial body 50 represents the shape of the implant. The medial/lateral geometry exactly represents the implant size and therefore allows the surgeon to position the femoral BCS cutting guide in the desired location prior to cutting bone. The trial 50 will be fixed in place with spikes or pins. The outer articular surface can be offset to make the body thicker and more rigid. This ensures the part retains the rigidity needed, and that it does not flex. This also provides feedback to the surgeons about the condition of the AP box cuts (previously done in surgical technique) and the ability of the implant to seat on the femur bone. The anterior and posterior square guides will allow passage of a drill followed by a chisel to remove and prepare bony tissue for the PS or BCS implant.

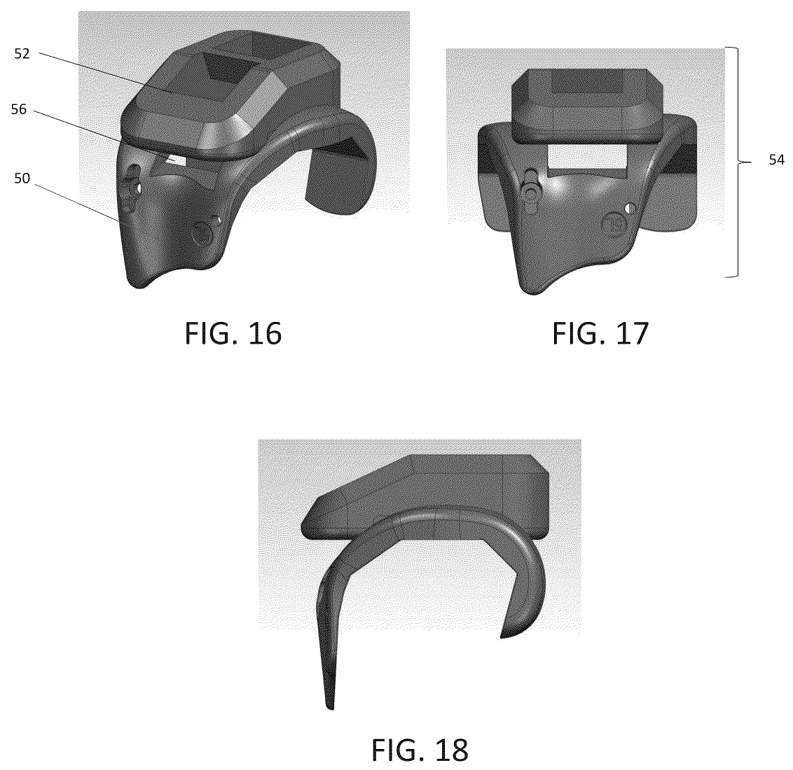

In the alternate embodiment of FIGS. 16-18, the trial body 50 and box guide 52 are united together as one instrument. This embodiment provides a slotted section 56 under the box guide 52 that allows for a saw cut required for the cruciate retaining (CR) canopy cut. Once the trial instrument positioned in the desired medial/lateral location (again, can be fixed in place with spikes or pins), the surgeon can then preform the needed resections for either the BCS, PS or CR implant.

It should also be understood that the femoral trial 50 and box cutting guide 52 may be separate pieces and attach together. They may be attached prior to use or during surgery.

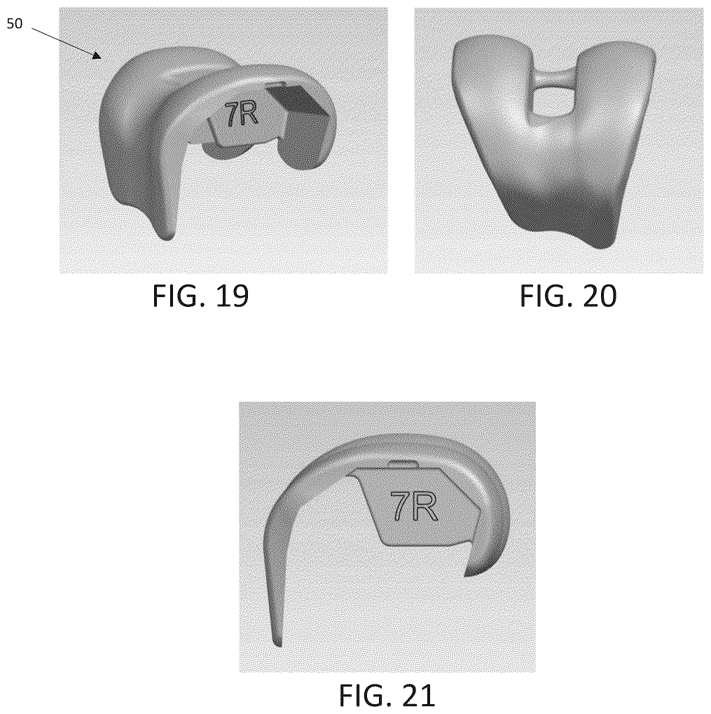

The further embodiment of FIGS. 19-21 contains the design of just the femoral trials 50 (CR, PS, BCS), without the box guide. These designs are representative of the implant and are meant to assess fit and function. They are 3D printed disposable parts. In some examples, that may be made from materials such as Somos PerForm or Rigid 10k or similar material. The trial contains all features of the implant geometry and contains a smooth outer articular surface.

The box cutting guide proposed is a low-cost disposable cutting guide. The current disposable concept is an improvement over other existing designs in terms of single use, disposable, low cost, material and because it meets the need of a heavy, expensive metal femoral trial. However, the disclosed designs can be customized, can be made on site, and can be lower cost options.

In summary, the use of 3D printing allows for the ability to design and manufacture connection features in one piece and provide recessed features within the block that would not be possible with standard manufacturing methods. The use of multiple biocompatible materials and modular spikes allow for the design flexibility to address features that require higher strength properties when under impaction loads.

What is claimed is:

1. A cutting guide, comprising:
a cutting block with an upper surface (12), a bone-facing surface (14), and a plurality of cutting slots (16);
at least one modular attachment spike (20) configured to cooperate with a mating feature (18) on the bone-facing surface of the cutting block.

2. The cutting guide of claim 1, wherein the mating feature comprises internal threads (26) that receive a threaded protrusion (28) of the spike.

3. The cutting guide of claim 1, wherein the mating feature comprises a female receiver (42) that receives compressible arms (40) of the spike.

4. The cutting guide of claim 1, wherein the bone-facing surface comprises an indentation (22) and the attachment spike comprises a subplate upper shoulder 24 that is received by the indentation.

5. The cutting guide of claim 1, wherein the cutting block is 3-D printed.

6. The cutting guide of claim 1, wherein the attachment spike is 3-D printed.

7. The cutting guide of claim 1, wherein the attachment spike is machined from metallic material.

8. The cutting guide of claim 1, further comprising at least one pin opening on the cutting block.

9. The cutting guide of claim 1, further comprising a femoral trial body.

10. The cutting guide of claim 9, wherein the femoral trial body is integrally formed with the cutting guide to provide a unitary instrument.

11. The cutting guide of claim 9, wherein the femoral trial body is provided separately from the cutting guide.

12. A method for preparing a patient's bone to receive an implant, comprising:
(a) securing one or more modular attachment spikes to a bone-facing surface of a cutting guide;
(b) securing the cutting guide onto the bone to be prepared.

13. A cutting guide, comprising:
a cutting block with an upper surface (12), a bone-facing surface (14), and a plurality of cutting slots (16);
at least one opening extending through the cutting block for securing the cutting block to a resected bone surface,
at least one gully (64) on the bone-facing surface.
